(19) United States
(12) Patent Application Publication
(10) Pub. No.: US 2008/0009443 A1
(43) Pub. Date: Jan. 10, 2008

(54) DIAFILTRATION SYSTEM TO FRACTIONATE PROTEIN MIXTURES

(75) Inventor: Howard E. Purdum, Raleigh, NC (US)

Correspondence Address: OBLON, SPIVAK, MCCLELLAND MAIER & NEUSTADT, P.C., 1940 DUKE STREET, ALEXANDRIA, VA, 22314, US

(73) Assignee: Throwleigh Technologies, L.L.C., Ball Ground, GA (US)

(21) Appl. No.: 11/683,029

(22) Filed: Mar. 7, 2007

Related U.S. Application Data
(60) Provisional application No. 60/779,425, filed on Mar. 7, 2006.

(51) Int. Cl. A61K 38/36; A61K 35/16; B01D 21/30; C02F 1/44; C07K 14/76

(52) U.S. Cl. 514/12; 210/137; 210/637; 530/362; 530/380

(57) ABSTRACT

The present invention provides a method for the separation of proteins on the basis of molecular weight by use of a multi-segment fractionation chamber based on the principles of diafiltration. In addition, the present invention provides an effective method for the concentration of plasma and recovery of fractions thereof that contain immune factors, clotting factors, and/or albumin. The present invention also provides a plasma diafiltration system and apparatus for the safe preparation of clotting factor concentrates that have broad and practical application to surgery or trauma patients, and patients lacking clotting factors due to inherited diseases or other complication.

### CROSS-REFERENCE TO RELATED APPLICATIONS

This application claims priority to U.S. 60/779,425, filed on Mar. 7, 2006, the entire contents of which is incorporated herein by reference.

### BACKGROUND OF THE INVENTION

1. Field of the Invention

The present invention provides a method for the separation of proteins on the basis of molecular weight by use of a multi-segment fractionation chamber based on the principles of diafiltration. In addition, the present invention provides an effective method for the concentration of plasma and recovery of fractions thereof that contain immune factors, clotting factors, and/or albumin. The present invention also provides a plasma diafiltration system and apparatus for the safe preparation of clotting factor concentrates that have broad and practical application to surgery or trauma patients, and patients lacking clotting factors due to inherited diseases or other complication.

### DISCUSSION OF THE BACKGROUND

Plasma is the straw-colored liquid that remains after all of the cellular components of blood have been removed. Consisting of water, electrolytes, various nutrients, immune factors and clotting proteins, plasma has many life-supporting functions. For this reason, plasma is often used for direct transfusion, primarily for cases involving massive blood loss. Many of the individual components of plasma can also be separated and used to treat a variety of diseases, with more than 100 such products now being produced by a multi-billion dollar, worldwide industry.

Thus, there is an immense demand for plasma and plasma products. However, it is not possible to obtain enough material to meet these demands. Although there has been some success in various synthetic techniques, the main source of plasma and plasma products remains the human donor. The overall donation process begins at the collection center. At this point, plasma is either separated from a whole blood donation, or obtained by apheresis, a process that takes only the plasma component of the blood from the donor. Some of the plasma is then used for direct transfusion, and some of the plasma is frozen and then thawed to obtain cold temperature insoluble proteins called cryoprecipitates. Most of the collected plasma, though, is sent to central processing facilities, where it is combined into large vats from which the individual components are then separated.

It has been found desirable for a wide variety of reasons to concentrate one or more components of multi-component materials. For example, in the biomedical field, it is often desired to increase the concentration of materials such as blood constituents, including plasma, immunoglobulins, fibrinogen and/or clotting factors, by removing water and/or other components of the material. In the pharmaceutical field, concentration of drugs or other materials produced in dilute liquid form or in solution is often required to produce an effective or commercially viable product. Food products such as condensed milk are also produced by means of material concentration processes. Material concentration processes also find application in the chemical processing industry, for example, in the removal of water from aqueous solutions, in the removal of organic solvents such as alcohols or alkanes from organic solutions, and the removal of inorganic solvents such as acids from in organic solutions. The concentrated materials may be reconstituted for use by addition of water, saline solution or other materials, or may be used or further processed in concentrated form.

Concentration of a material may be desirable in order to minimize the expense and space requirements related to storage and transportation of the material. For example, the storage and shipment of blood products typically requires expensive refrigeration equipment. The effective capacity of available equipment can be increased by minimizing the volume of the shipped or stored products through material concentration. Increased availability of blood products can save lives in emergency situations such as natural disaster or war, and can provide substantial economic savings in nonemergency applications. Concentration of a material may also be desirable in order to enhance or alter the properties or therapeutic effects of the material. For example, fibrin glue formed by concentration of fibrinogen and other components in blood plasma has found increasing application in the repair of traumatized biological tissue. The concentration of a material also may assist in, or enhance the efficiency of, additional processing of the material. For example, concentration of blood plasma reduces the volume of material to be treated in subsequent decontamination and fractionation steps, thereby reducing the time, expense and equipment requirements for these processes.

Material concentration can also enhance the detection of contaminants in a product by increasing the concentration of the contaminants, thereby rendering them more easily detectable. Previously known material concentration methods have been found to be less than fully successful for many applications. In particular, temperature-sensitive materials are often damaged by known material concentration methods. For example, forced evaporative and distillation methods of concentration, which typically involve the application of heat to the material to be concentrated, can irreversibly denature proteins or otherwise damage the product. Previously known cryoprecipitation methods of concentration, which typically involve freezing the entire quantity of material to be concentrated, can likewise damage temperature-sensitive products. Previously known filtration methods of material concentration typically suffer inefficiencies due to clogging of the filter media, necessitating frequent replacement or cleaning of the filter. Previously known methods and systems for concentrating also suffer from low yields and inefficiencies. For example, pump and line losses often consume a substantial quantity of concentrate in known methods and systems.

Thus it can be seen that a need yet exists for a method and system for concentrating temperature-sensitive materials, which method reduces or eliminates damage to the materials, reduces inefficiencies and increases yield.

It is also known to separate some proteins from blood plasma by cryoprecipitation. The basic principle of cryoprecipitation is that some plasma proteins agglomerate when frozen, and then remain agglomerated when thawed if the temperature is kept sufficiently low, no more than 4° C. This technique can thus be used to separate certain proteins, such as Factor VIII, fibrinogen, and von Willebrands factor, from bulk plasma.

Conventional cryoprecipitation techniques, however, suffer from long processing times and poor yields; these limits are indeed some of the prime motivations for the concentrator. It is therefore desirable to develop a cryoprecipitation technology specifically for concentrated plasma. It is also known to separate and/or purify materials by chromatography. The underlying principle in chromatography is that different materials diffuse through different media at different rates. These differences in rates thus provide a means of separating the various components of complicated mixtures. Such separations are commonly used to identify individual components, such as toxins or other unknowns, and to prepare commercially valuable fractions of known mixtures, such as blood plasma. In conventional chromatography, the target materials of interest are often organic compounds, which can be in liquid or gaseous forms. The target materials are usually dissolved in a solvent, such as alcohol. The media typically consist of absorbing materials, such as paper or gels.

The overall process amounts to a progression of equilibrium states (K. Hostettmann et al, Preparative Chromatographic Techniques, Springer Verlag, 1998, which is incorporated herein by reference), during which the material to be separated reaches equilibrium with the media and the solvent. The ideal situation is that the flow rates and the relative absorption strengths are balanced well enough to resolve the components.

There are, however, four major factors that act against these ideal conditions. First, the sample may be so large that the starting conditions are not well defined, i.e., part of the sample may be subject to solvent motion, while the rest of the sample sees no treatment, Second, molecular diffusion of the solute under the action of the increasing concentration gradient tends to spread the material in all directions. Third, eddy diffusion due to irregularities in the media can also spread the solute in all directions. Fourth, the resistance of the media to mass transfer can hinder local equilibration. The net effect of these, and other lesser factors, is to spread the components (i.e., the components migrate as broad, possibly overlapping bands as opposed to narrow, resolved bands), thereby reducing the resolution of the system.

To overcome these problems, a number of alternatives are available. These techniques, which include the use of high pressure, rotation, ion exchange, affinity, etc., are often quite successful, but are expensive, complicated, and require long processing times. These problems are particularly severe for high molecular weight components, such as blood plasma proteins.

Nevertheless, chromatography is still the preferred technique for isolating plasma proteins. Compared to the older, but still practiced, Cohn, or cold ethanol, fractionation procedure, chromatography yields greater resolution and less protein damage. For these reasons, new facilities, such as the Australian national unit, are designed for chromatography. Even in this state-of-the-art facility, however, the process is still quite involved and lengthy. For example, a given batch of plasma requires approximately 3 months for complete processing. This very long time is in fact the underlying problem behind recent shortages of various immunoglobulins in the United States, shortages so severe that FDA has relaxed some safety standards.

Thus, there also remains a need for improved techniques for the separation and/or purification of materials found in blood plasma.

One promising method of improving the processing of temperature-sensitive material, including plasma, is provided by U.S. Pat. No. 6,808,638. The present application is an extension of the seminal plasma concentration work set forth in U.S. Pat. No. 6,808,638. This patent describes a means of concentrating plasma (or any other heat sensitive compound) by freezing out pure ice (with ultrasonic assist), and then removing this ice. This technology concentrates all solutes, including salts. The concentrated material can then be easily stored, handled, frozen, thawed, and used for further manufacture.

In the practice of medicine, it is quite desirable to be able to administer concentrated plasma directly to patients, primarily those suffering trauma, undergoing high blood loss surgerical procedures, such as hip replacement, or suffering loss of clotting factors due to inherited disorders or other diseases. The underlying problem here is that unconcentrated plasma contains only several milligrams of clotting protein(s) per liter of solution, and is thus quite dilute. For this reason, severely bleeding patients can quickly be overcome with excess liquid before effective clotting is obtained, and therefore bleed to death.

The most common alternative approach to direct plasma transfusion is to administer clotting factor concentrates. In particular, there is currently a great deal of interest in activated recombinant factor VII: rFVIIa. Unfortunately, rFVIa does not satisfy all plasma transfusion needs, can be hazardous to patients with head injuries, and is extremely expensive.

Therefore, a critical need still exists for the development of effective methods for the concentration of plasma. In particular, there exists a critical need for the development of safe clotting factor concentrates that have broad and practical application to trauma patients.

Further, a demand continues to exist for safe and effective protein fractionation techniques that permit fractional isolation and resolution that may be tuned to a desired level of purity and/or to permit recovery on the basis of molecular weight.

### SUMMARY OF THE INVENTION

It is an object of the present invention to provide an apparatus for protein fractionation having:

a) a plurality of segments; and

b) a membrane at the base of each segment,

wherein each segment has a circular shape with a diameter to height ratio ranging from 10:1 to 20:1,

wherein each segment contains a broad, flat rotor induces complete mixing,

wherein each segment comprises a pressure affecting means,

wherein each segment has an input prior to said membrane and an outlet after said membrane,

wherein each segment is coupled to the input of the subsequent segment through its outlet wherein a pump or regulator system is positioned downstream of the membrane to maintain a pressure difference across the membrane,

wherein the molecular weight cut-off of each membrane is lower than the membrane in the segment upstream thereof.

It is an object of the present invention to provide such an apparatus for plasma fractionation. Specifically the apparatus for such a method would have either three or four chambers.

It is another object of the present invention to provide a method of fractionating plasma by

a) adding plasma to the first segment of the chamber of the present invention;

b) applying a pressure to the solution in the first segment to facilitate transfer of solutes across the membranes;

c) stopping the fractionation after an appropriate time to reach a final transfer equilibrium; and

d) recovering samples from each of the first, second, and third segments,

wherein each of (a)-(d) are performed under sterile conditions.

It is yet another object of the present invention to provide fractional concentrates recovered from the three segments of the three-segment chamber for plasma fractionation following the foregoing fractionation method. In the first segment, the fraction is a immune factor and clotting factor concentrate, the second segment contains an albumin concentrate, while the third segment contains small plasma proteins and sugars.

It is yet another object of the present invention to provide fractional concentrates recovered from the four segments of the four-segment chamber for plasma fractionation following the foregoing fractionation method. In the first segment, the fraction is an immune factor concentrate, the second segment contains a clotting factor concentrate, the third segment contains an albumin concentrate, while the fourth segment contains small plasma proteins and sugars.

It is still another method of the present invention to provide a method of promoting clotting in a subject in need thereof by administering to the subject an effective amount of the clotting factor concentrate recovered by the fractionation method above.

And, yet another object of the present invention is a unique stirring capability that will avoid activating the clotting sequence while still preventing polarization.

The above objects highlight certain aspects of the invention. Additional objects, aspects and embodiments of the invention are found in the following detailed description of the invention.

### BRIEF DESCRIPTION OF THE FIGURES

A more complete appreciation of the invention and many of the attendant advantages thereof will be readily obtained as the same becomes better understood by reference to the following Figures in conjunction with the detailed description below.

FIG. 1 shows a flow chart of the overall process.

FIG. 2 shows the first separation module, including the means of providing pressure, the stirring mechanism, and the membrane support grid. The steel mesh (or screen) is bound by a steel ring to maintain its shape under pressure: the ring keeps the mesh flat.

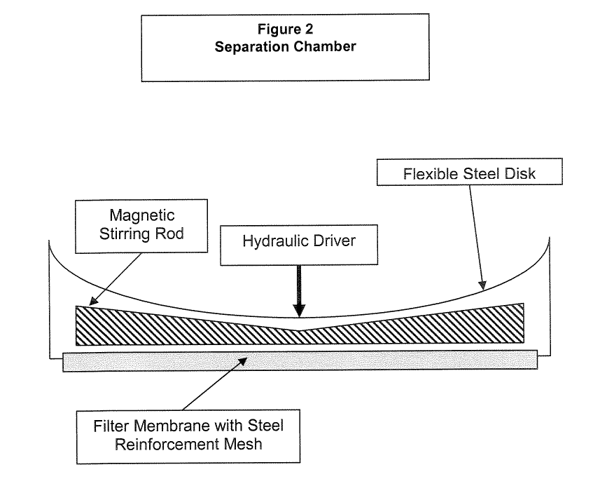

FIG. 3 shows a graphical depiction of one separation module and couplings. As described herein, the separation module (i.e., segment) can be daisy-chained at will, coupling the outlet from one unit to the input of the next. The only requirement is that there is a pump or regulator system downstream of the membrane so that the pressure difference across the membrane is maintained. As shown, a peristaltic unit can actually apply some vacuum, further aiding the process.

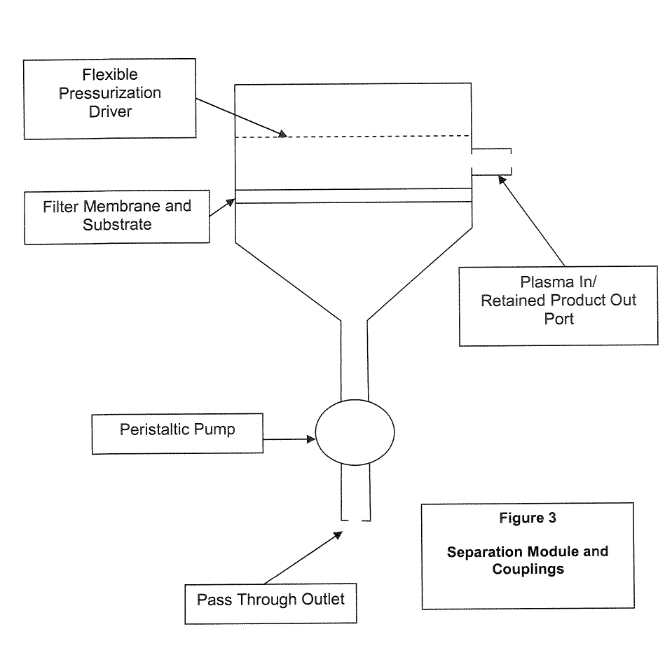

### DETAILED DESCRIPTION OF THE INVENTION

Unless specifically defined, all technical and scientific terms used herein have the same meaning as commonly understood by a skilled artisan in enzymology, biochemistry, cellular biology, molecular biology, and the medical sciences.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

The present invention address the critical need that exists in the art for safe and effective protein fractionation techniques that permit fractional isolation and resolution that may be tuned to a desired level of purity and/or to permit recovery on the basis of molecular weight. Further, the present invention addresses the critical need still existing in the art for the development of effective methods for the concentration of plasma. In particular, the present invention addresses the critical need still existing in the art for the development of safe clotting factor concentrates that have broad and practical application to surgery or trauma patients, and patients lacking clotting factors due to inherited diseases or other complication.

To satisfy the foregoing, the present invention is based on a modified diafiltration system to fractionate and concentrate protein mixtures, including plasma. Specifically, the present invention provides new diafiltration chamber technology including a new diafiltration apparatus. In addition, the present invention provides a method of fractionating protein mixtures, including whole plasma obtained from apheresis or whole blood. The present invention also provides concentrated fractions of plasma proteins (e.g., immune factors, clotting factors, albumin, and small proteins and sugars) obtained by the diafiltration method described herein.

Beginning with the plasma protein details, the first concern is that the elevated salt content due to concentration will raise the osmotic pressure and thus dehydrate the cells. One alternative is dialysis, but this approach will cause dilution of the proteins, which is counter to the concentration step. It is therefore necessary to perform the salt removal without accumulating water in the material being treated.

The next concern is that the increased concentration of albumin in the concentrated plasma sample will raise the oncotic pressure to dangerous levels, noting that the administration of albumin concentrate to burn victims must be carefully monitored. In addition, it must also be noted that albumin comprises about ⅔ of the plasma protein volume, and it is not required as part of the clotting process. In this regard, albumin is often added to clotting protein concentrates, but only to serve as a stabilizing agent.

The net result is that to transfuse the concentrated plasma directly, the salt concentration must be reduced to physiological levels (i.e., approximately 0.9 wt %), and the albumin must be reduced as greatly as possible or removed altogether, without damaging or greatly reducing the clotting proteins.

Thus, the underlying principle behind this technology is that protein solutions can be separated by diafiltration. To enhance this process, the solution is subjected to elevated pressure on the source side, thereby driving the solutes more rapidly across the membrane. In addition, the solution is often stirred to prevent the solutes from accumulating near the membrane, in a process called “polarization,” and thereby slowing the transport across the membrane. “Diafiltration” is a technique that uses ultrafiltration membranes to completely remove, replace, or lower the concentration of salts or solvents from solutions containing proteins, peptides, nucleic acids, and other biomolecules. The process selectively utilizes permeable (porous) membrane filters to separate the components of solutions and suspensions based on their molecular size. An ultrafiltration membrane retains molecules that are larger than the pores of the membrane while smaller molecules such as salts, solvents and water, which are 100% permeable, freely pass through the membrane. One example of this technology is the Millipore “Amicon” stirred cell system, while another is exemplified in U.S. Pat. No. 7,001,715.

As evidenced by either of the aforementioned diafiltration apparatus, the standard diafiltration apparatus is a two chamber system in which the solution is forced through the membrane via gas pressurization.

The standard diafiltration methods suffer from several downfalls, including ease of use and the ability to resolve plasma while properly achieving physiological salt levels. Accordingly, what is necessary is a new separation chamber to process plasma.

Although the present invention is described below largely for separation of plasma proteins from plasma samples, this is merely for convenience as the invention was initially derived from plasma samples. However, as would be readily apparent to the skilled artisan, the present invention may be extended to application with protein mixtures obtained from various sources, including (but not limited to): recombinant sources (e.g., bacterial cultures), manufacturing lines (e.g., synthetic proteins), and biological or medicianl specimens.

In accordance with the present invention, the new separation chamber will be comprised of at least three segments. The segments of the chamber are further described below.

In the plasma separation chamber of the present invention, the plasma will be fed into the first segment. In an embodiment of the present invention, the plasma will be added to the first segment along with sufficient water to wash out the salts.

The purpose of this first segment is to retain the clotting proteins. Since, the molecular weight of albumin is about 68 kiloDaltons and it is desired that the albumin pass through to the second chamber, the first segment will retain everything above this size when the appropriate size molecular weight cut-off membrane is employed.

Of course, the cutoff is not precise due to manufacturing difficulties and the normal range of albumin sizes in the body. Furthermore, for practical reasons, it is desirable to have a system with pores somewhat above the cutoff- otherwise, the process takes too long. This is not a problem, however, because some albumin can remain without any consequences. As such, the molecular weight cut-off of the membrane bounding the first chamber should range from 70-140 kDa, preferably 100-120 kDa, and more preferably from 115-120 kDa.

By using a molecular weight membrane it is possible to retain substantially all of the clotting proteins. Due to the close similarity of molecular weight of clotting factor X with that of albumin, it may be difficult to resolve these two proteins and clotting factor X may pass through the membrane bordering the first segment of the chamber. However, clotting factor X is not critical to the clotting ability of the fraction recovered from this segment.

Accordingly, as used herein, the term “substantially all” embraces two independent concepts. First, as used herein, the phrase “substantially all” is intended to mean that at least 90%, preferably at least 95%, more preferably at least 97.5%, and most preferably at least 99% of all protein types (specifically clotting proteins) having a molecular weight greater than the molecular weight cut-off of the membrane bounding the segment are retained in the first segment. Second, as used herein, the phrase “substantially all” means that at least 90%, preferably at least 95%, more preferably at least 97.5%, and most preferably at least 99% of the protein quantity of the protein types (specifically clotting proteins) retained in the first segment are retained in the first segment.

In order to drive the solution through the chamber of the present invention, it is preferred that a pressure is applied to the first segment. In one embodiment of the present invention, the pressure is applied by gas by conventional means. In an alternate method of the present invention, the pressure is applied by a hydraulic mechanism (e.g., a hydraulic driver). Of course, depending upon the pressure source selected the chamber will reflect the specific demands of that pressure source. In other words, where the pressure to be applied is gas pressurization, the chamber walls of the first segment include a gas inlet port. The gas inlet port is preferably at or near the top of the first segment to ensure that the pressure is applied. Where hydraulic pressurization is selected, the first segment is equipped with a motor to drive a hydraulic pressure seal, which makes a tight seal to the chamber wall while the hydraulic pressure forces the plasma sample through the membrane bounding the segment. Further where hydraulic pressurization is used, it is contemplated that said pressure is exerted upon a sealed and bound bag containing the plasma components.

Although the pressure in the separation modules (i.e., segments) may be maintained a low pressures typical for standard diafiltration techniques (e.g., 30-100 psi or 50-75 psi), in the present invention it is preferred that a steel mesh (or screen) is bound by a steel ring to maintain its shape under pressure upon which the membrane is affixed (infra). The ring maintains the mesh flat under elevated pressures, thus preventing the mesh from bulging out from the center. Since pressure and surface area of the membrane helps to facilitate the speed at which samples may be fractionated, which is important with blood/plasma specimens where time-based limitations are imposed for use once isolated, where the metal mesh is present, the pressure may be elevated to 200 to 1000 psi, inclusive of any integer and subrange there between. Ranges that warrant further specific mention include 200 to 500 psi and 250 to 350 psi.

In an embodiment of the present invention, the first segment is equipped with a stirrer to prevent the solutes from accumulating near the membrane (i.e., to polarize the solutes), which would slow transport across the membrane.

Each of the second, third, and subsequent segments of the chamber are preferably structurally identical to the first segment described above, but for the membrane bounding the segment. However, it is within the scope of the present invention for the second, third, and subsequent segments to be modified to suit the individually needs of the separation. Such a modification could entail differences in applied pressure values, pressure application device type or form, membrane composition, presence or absence of the mesh, materials used to construct the segment's structure, dimensions of the segment, presence or absence of a rotor, etc.

The second segment of the chamber of the present invention is bound by a membrane that retains substantially all of the albumin. Again, albumin is a 68 kDa protein, therefore, the membrane bounding this segment should range from 40 to 65 kDa, preferably 45 to 60 kDa, more preferably from 50 to 55 kDa.

Of course, to prevent solute build up on the membrane bounding this segment, it is permissible that this segment be fixed with a stirrer to polarize the solutes.

The third and final segment of the chamber of the present invention is bound by a dialysis membrane with a molecular weight cut-off ranging from 5 to 15 kDa, preferably 5 to 10 kDa that passes salts and retains everything else (larger proteins, sugars, etc.).

The proteins above albumin also include immune factors which are quite valuable as feedstocks. While not of immediate use for transfusion, they are too valuable to throw out. Furthermore, blood banks do not want to have multiple tracks, and therefore all plasma should be treated the same way. Thus, we wish to fractionate all plasma. The plasma that is not used for transfusion will then be sent to the fractionators, noted above. The advantage for the fractionators is that the fractionated plasma will already be partially separated, thus saving time and expense at the factory.

Thus, in an embodiment for fractionation of plasma that is alternative to the above-described three-segment chamber, the present invention provides a four-segment chamber that permits fractionation of immune factors. The four-segment chamber is identical to the three-segment chamber described above, but for the addition of a fourth segment preceding the “first” segment above. Thus, the first segment of the three-segment chamber (clotting factors) becomes the second segment of the four-segment chamber, the second segment of the three-segment chamber (albumin) becomes the third segment of the four-segment chamber, and the third segment of the three-segment chamber (salts) becomes the fourth segment of the four-segment chamber.

In the four-segment chamber of the present invention, the first segment is for isolation of immune factors. Fibrinogen is the largest of the clotting factors with a molecular weight of about 250 kDa, whereas the immune factors typically have a molecular weight on the order of about 1 MDa. Therefore, to fractionate the immune factors from the clotting factors, a first segment with a membrane having a molecular weight cut-off ranging from 300 kDa to 500 kDa, preferably from 350 kDa to 450 kDa is employed.

Within the scope of the present invention, it is contemplated that the flat membranes bounding each segment may alternatively (individually or collectively) incorporate hollow fiber technology.

With respect to the flat membranes, in an embodiment of the present invention the membranes may be traditional membranes provided by any commercial supplier (e.g., Millipore or Fresenius). The various membranes are organic based, with proprietary additives selected by the manufacturers. There is no limitation here, as long as the membranes are acceptable by FDA for blood contact. Where the application is for non-blood protein mixtures, any membrane may be employed.

The shape of the membranes is not restricted. Generally, it is preferred that the membrane be of a circular form. This form is favored due to the uniformity of pressure across the surface of the membrane. When the membrane is circular, the membrane simply be placed on the base of the segment over the tope of metal mesh (supra) or opening at the bottom of the segment, as appropriate, without additional structural assembly to secure the membrane in place, although such an assembly may certainly be used. However, the present invention also embraces other membrane shapes, including (but not limited to): square, rectangular, ovular, pentagonal, hexagonal, etc. Indeed, square or rectangular shapes may be preferred from a cost-effectiveness position when the samples to be fractionated are of a recombinant or manufacturing line. In the case of non-circular shapes, the pressure applied to the chamber segment may not uniformly apply across the surface of the membrane. In this case, it is preferred to either alter the structure of the segment to provide fasteners to secure the membrane. Alternatively, the non-circular membranes may be affixed to the segment by use of other binders (e.g., biologically acceptable adhesives).

As described above, in an embodiment of the present invention, the membranes of the present invention may incorporate a metal screen in a grid layout to support the membrane, thus enhancing the load of the membrane. Further, the metal screen would keep the cost to a minimum (cheaper than fibers, without the cost of a great deal of substrate). The advantage of this embodiment is that the grid will reinforce the substrate such that it will fail only by shear over the grid lines, versus the much easier bursting of an unsupported membrane. As with the chamber assembly itself, stainless steel is commonly specified in medical devices and is preferred for the structure of the screen. However, any FDA accepted metal may be used for the chamber of the present invention. Further, where the chamber is to be used for fractionation of non-medicinal specimens (e.g., recombinant protein samples), any material may be used to for the structure of the chamber and the mesh although stainless steel is still generally preferred.

As used herein the term “first membrane” means the membrane between the first and second segments of the chamber. Similarly, the term “second membrane” means the membrane between the second and third segments of the chamber, and so on.

In a particular embodiment of the present invention, the individual segments of the chamber are short and wide to maximize membrane exposure and minimize the average distance to the membrane for the solute to traverse. In an embodiment of the present invention, the individual segments of the chamber have a ratio of the width (diameter) to the height ranging from 10:1 to 20:1, preferably 12.5:1 to 17.5:1, more preferably 15:1. In one embodiment, the individual segments of the chamber have a diameter that is about 25 to 35 cm (preferably about 30 cm) and a height of about 2 to 3 cm.

Obviously, the present invention is not to be limited to the centimeter-scale. The present invention finds utility in large industrial-scale applications as well as small fine-scale application. For scaling, (1) at the high end, the limitation is that the farthest point from the membrane must be close enough for reasonable chance of transport—the closer, the better, being constrained by cost: a great deal of membrane is necessary for extremely thin layers, and (2) at the low end, the limitation is the strength of the rotor—it will break if it is too small. Accordingly, the size may be adjusted so long as the height does not become so large as to prevent effective membrane transport (large size) or the rotor does not become vanishingly small (small size).

Coupled with this geometry, a broad, flat rotor that fills much of the chamber volume is preferably used (preferably at least 75%, at least 80%, at least 85%, at least 90%, or at least 95%). Preferably, this broad, flat motor is a slow moving rotor, which will thus rapidly turn over the chamber volume, while minimizing the shear that would otherwise damage the proteins. The rotor must be able to induce complete mixing. As stated above, the rotor should fit inside the separation module. This rotor will thus be much longer than conventional magnetic stirring devices, although it will be magnetically coupled to maintain a sealed system.

Unlike conventional rotors (which mix by forming a vortex), the rotor of the present invention will turn much more slowly to prevent protein damage. For adequate mixing, the rotor of the present invention is shaped much like a propeller. Given the low speed (unlike high speed, vortex forming units), the mixing can be effective over only a few rotor heights, depending on speed. This will limit the geometry of the chamber design favoring circular structures where mixing can be effectively controlled throughout the sample as opposed to non-circular shapes where fluid mixing is locally inefficient (e.g., corners of a square chamber). As such, the chamber of the present invention and the rotor used therein is unlike any competing units.

As stated above, with the unique rotor of the present invention, low speeds are preferred to prevent protein damage. Similarly, low speeds are desired to prevent initiation of the clotting sequence where the proteins to be fractionated are plasma proteins. Therefore, the speed is preferably maintained to no more than 50 rpm, preferably no more than 40 rpm, more preferably no more than 30 rpm. With respect to a lower limit on the rotor speed, the rotor must be able to effectively facilitate complete mixing of the sample contained in the separation module. Typically this may be achieved with a minimum rotor speed of 10 rpm, although lower speeds are contemplated so long as mixing can be achieved.

In a desired embodiment of the present invention is that each segment of the chamber is equipped with a sensor to determine when physiological salt limits have been reached. Other information that may be obtained from the sensor include when transfer equilibrium has been reached.

Within the present invention, it is preferred that the plasma to be added to the first segment be of human origin. Methods of obtaining and harvesting human plasma are well-known to one of skill in the art and, therefore, are not repeated herein.

It should be noted that the method and chamber described herein are not limited to human plasma. The method and chamber of the present invention may also be used for plasma obtained from any other source, in particular a mammalian source. A non-exhaustive list of sources of plasma for the present invention include: human, horse, dog, cat, sheep, cattle, pig, primate, and mouse.

Also within the present invention, it is contemplated that one or all of the methods performed in each segments, respectively, may be performed at a temperature below ambient temperature. However, it is to be understood that it is also possible to perform each step, individually or collectively, at ambient temperature or up to 30° C., so long as the integrity of the proteins of interest are preserved. For fractionation of proteins of medicinal import (e.g., plasma), the FDA specifies the appropriate temperature ranges, which are typically low to prevent bacteria growth and to preserve the integrity of the plasma) proteins.

Since the present invention relates to fractionation of plasma for administration to humans or other mammalian sources, the plasma and all objects that it contacts (whether in whole or fractionated form) shall be sterile. Methods of sterilization are well known to the skilled artisan and therefore are omitted from extensive discussion herein.

The present invention also provides a method of fractionating plasma by adding plasma to the first segment of the chamber described above, applying a pressure to the solution in the first segment to facilitate transfer of solutes across the first and second membranes; stopping the fractionation after an appropriate time to reach a final transfer equilibrium; and recovering samples from each of the first, second, and third segments.

As stated above, the plasma to be added to the column may be from any source, but is preferably mammalian in particular human. Further, the plasma sterile and all steps should be conducted so as to maintain sterility at each step.

It is contemplated that one or all of the steps performed in the method of the present invention, respectively, may be performed at a temperature below ambient temperature to preserve the integrity of the plasma proteins. Generally, for protein fractionation the temperature is desired to be maintained at or around 4° C. However, it is to be understood that it is also possible to perform each step, individually or collectively, at ambient temperature or up to 30° C.

In the method of the present invention, the pressure to be applied to the solution in the first segment may be gas pressurization or hydraulic pressurization (see above) and may be performed with or without mixing in each segment of the chamber.

As used herein the phrase “stopping the fractionation after an appropriate time to reach a final transfer equilibrium” means that no further transfer of solute can be detected across either of the first or second membranes.

Methods of recovering the samples from the various segments of the chamber are not limited and would be readily appreciated by the skilled artisan. As such, the recovery methods are described herein.

In the present invention, it is contemplated that samples recovered from each of the first, second, and third segments may be further concentrated. One example of a further concentration method is cryoconcentration described in U.S. Pat. No. 6,800,638 (incorporated herein by reference in its entirety). Alternative concentration methods are also contemplated and embraced by the present invention.

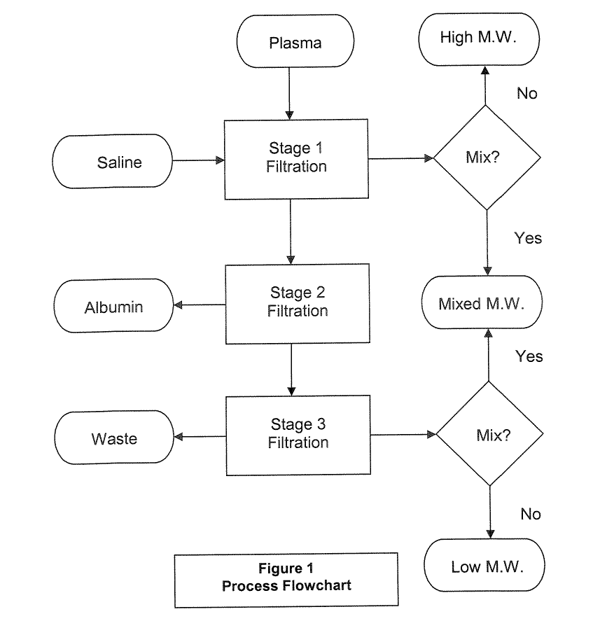

FIG. 1 of U.S. Pat. No. 6,808,638 shows a cryoconcentration unit—no membrane, no pressure. The overall intent here is that the present invention uses the fractionation unit (e.g., chamber) as a preparation step for the cryoconcentration device (U.S. Pat. No. 6,808,638). The reason here is that it is now possible to fractionate before concentrating—otherwise, the plasma will become too viscous to separate. For example, in practice, it is desirable to concentrate the plasma protein fractions about 4:1 (alternatively 2:1, 3:1, 5:1, 6:1, including all ranges and subranges there between) so as to cut the total fluid level to an acceptable volume.

By way of example, the present inventor provides the following illustration using a three segment chamber. In the case of apheresis plasma, the starting volume will typically be about 600 ml; from a whole blood donation, the starting volume will be about 150 ml (which is why blood banks prefer apheresis). Although the process applies to plasma collected by both techniques, the following illustrates the use of the apheresis example. Although data are not final, approximately 5 dilutions are needed for diafiltration. Therefore, 3 liters of saline will be mixed with the plasma, making it quite dilute at first, and therefore easy to filter. This can be done once in a closed system (i.e., batch wise processing)—no connections need to be made after filling, although this could be an option. The filled assembly is then be placed into the matching cavity of the pressure vessel. The vessel is then closed, and a hydraulic piston (or other driver) applies pressure to the chamber. The key features here: (1) no gas to handle, (2) the fixed walls bear the pressure (instead of the plastic chamber walls, therefore the plastic walls can be thin and cheap, and (3) if the plastic wall should break, the chamber will contain any leaks (versus a spray of possibly HIV contaminated plasma in the case of gas pressurized units—extremely dangerous indeed).

Also, the stirring rotor will prevent polarization here. Key features: the chamber is designed to be flat, wide and shallow—essentially like a pie plate. This arrangement provides maximum surface area at the base for high throughput, a thin fluid layer so that the fluid has easy access to the filter (again for high throughput), and minimum height change from start to finish (therefore minimum flexure required for operation, thereby minimizing the seal loading while ensuring constant pressure—envision a cymbal shape). The rotor will fit inside this chamber. This rotor will thus be much longer than conventional magnetic stirring devices, although it will be magnetically coupled to maintain a sealed system. Unlike conventional rotors (which mix by forming a vortex), this rotor will turn much more slowly to prevent protein damage. For adequate mixing, the rotor of the present invention is shaped much like a propeller.

When this process is finished, the concentrate in the first segment of the chamber is reduced in albumin content, as well as salts. Specifically, the salt will be about 1 or so of the starting level, depending on the filter quality, etc. The actual salt concentration will be measured by a conventional conductivity meter. Note that while extremely high salt concentrations can damage proteins, lower salt concentrations are relatively benign—no damage at this level. The albumin and salts are passed on to the second segment of the chamber where the process is repeated, but a lower molecular weight cut-off membrane is used to bound the second segment, preferably to retain albumin in the second segment, while the salts are passed on to the third segment of the chamber.

The cryoconcentration process can thus proceed on the concentrate recovered from the first segment (or subsequent segments individually) until the physiological 0.9% level is reached. As an added benefit, reducing the salt concentration before freezing the proteins for storage (plasma products can be stored for years with little loss of quality) helps to preserve the product: a key factor in freeze damage is locally excessive salt concentrations, therefore, reducing the salt to ¼ or so reduces the freeze damage to ¼ or so. Also, salt removal is a major problem in fractionation, so having much of the salt removed ahead of time is a great help.

The present invention also provides for fractional plasma concentrates, where these concentrates are recovered from the first, second, and third segments of the chamber. As stated above, from the first chamber the clotting factors may be recovered, from the second chamber albumin may be recovered, and from the third chamber small plasma proteins and sugars may be obtained.

Since no separation is perfect, there will be some, minimal overlap in the various fractions. With this in mind, in an embodiment of the present invention each fraction is substantially pure. In this context, the term “substantially pure” means that in the fraction at least 70%, at least 80 wt %, at least 90 %, or at least 95% of the solute contained in that fraction is the desired component on a plasma protein molecular weight basis (e.g., at least 70 wt % of the solute recovered from the second segment is albumin).

It is preferred that each fractional plasma concentrate recovered from the chamber of the present invention or by the method of the present invention be at physiological salt concentration, which is recognized to be 0.9 wt %. If the target salt concentration is not reached common means of adjusting the same may be employed.

If necessary to enhance stability of the clotting factor concentrate it is contemplated that albumin may be added back to the fraction recovered from first segment of the chamber.

As stated above, depending upon the molecular weight cut-off of the first membrane, factor X may be lost from the clotting factor concentrate and will appear in the albumin concentrate. Since factor X is not essential for effective clotting, the loss of this factor is of no consequence. However, where it is desired to recover a substantially pure albumin concentrate, the molecular weight cut-off of the first membrane may be selected such that factor X and some albumin are retained in the clotting factor concentrate of the first segment.

The fractional concentrates of the present invention may be stored for any desired length of time. Although the shelf life at 4° C. of the clotting factor concentrate may find limits, this sample may be prepared for long term storage or for rapid use in field medical facilities in any known manner. For example, the clotting factor concentrate may be lyophilized or flash frozen. Preferably, the clotting factor concentrate is flash frozen because lyophilization may lead fibrinogen breakdown, thus resulting in a need for administration of more clotting factor to a patient in need thereof.

In recognition of the fact that the plasma fractional concentrates have broad applications, FIG. 1 of the present invention details an overall process flowchart based on the use of a three-segment chamber. For example, it is possible to combine the output from Stages 1 and 3 to provide nutrients, salts, etc. to the highest molecular weight (M.W.) proteins. Alternatively, these components may be held separately. This strategy is useful when the fractionation method/apparatus is to be used in a military field hospital, for example, where resources are at a premium and waste must be prevented at all cost. In this instance, there is a need to add back the nutrients, especially where nutrient supplies are limited. However, in suburban hospitals and/or with manufacturing processes, nutrient supplies are plentiful and may not need to be added back to the clotting fraction. Obviously, this alternative mixing scheme also applies to the four-segment chamber, where the nutrients from the fourth segment may be added back to either the immune factors or the clotting factors, or both.

The present invention also provides a method of promoting clotting in a subject in need thereof by administering to a subject in need thereof an effective amount of the clotting factor concentrate.

Within this embodiment, a preferred mode of administration is intravenously. Therefore, the method of delivery of the clotting factor concentrate is by direct intravenous injection, by piggybacking on an existing intravenous line. An alternative administration route is by mixing the concentrate with thrombin and spraying/pouring/extruding on open wound areas to induce hemostasis (clotting). A suitable device of application on open wound areas is readily available to the skilled artisan. An example of which would be a two-chambered syringe or applicator in which thrombin is preserved in one chamber while the clotting factor concentrate would be housed in the second chamber of the two-chambered syringe or applicator. By using this two-chambered syringe or applicator it is possible to maintain the two factors in isolation until the moment and site at which hemostasis is desired.

In the case of plasma samples, the samples acquired from subjects are either whole blood or apheresis. Therefore, typically, prior to fractionation in accordance with the present invention the samples are prepared either by: an apheresis kit (apheresis sample) or by centrifugation (whole blood sample), to remove any undesired agents or cells (e.g., white or red blood cells). In either event, an anti-coagulant (for example, sodium citrate) is added to the sample to prevent premature clotting prior to fractionation.

The present fractionation method and apparatus may be used in a continuous or batch-wise manner, depending upon the use and/or source of the sample to be fractionated. Where the sample is plasma for use in blood single unit transfusion the method is only to be performed in a batch-wise manner to comply with FDA mandates and to decrease the risk of infection to the recipient. However, where the sample is non-blood or where the source is a recombinant or manufacturing line, the method and/or apparatus may be adapted for continuous processing by steady input of sample into the intake port of the first segment.

Where the apparatus of the present invention is to be used for plasma separation, it is important that sterility be maintained throughout the chamber. Therefore, prior to use of the system, the chamber is preferably irradiated with gamma-radiation.

The term “effective amount” as used in this context is understood to be a clinical term that will vary based upon the patient's physical characteristics (e.g., height, weight, age, etc.), physical condition (e.g., extent of injury), and response to treatment. Therefore, this term is not intended to be limited to any specific dosage. However, in a preferred embodiment, the clotting factor concentrate is packaged into unit dosages. As per the source of the plasma sample, the units will be done according to collection, that is, one unit of donor plasma (whole blood or apheresis) makes one unit of product. Single donor systems are mandated by FDA to avoid cross contamination, i.e., it is better to have a single contaminated unit infect only one patient instead of pooling the product and possibly infecting others. However, where the sample is provided from recombinant or manufacturing lines, the unit dosage may be determined based on the nature and identity of the protein(s) to be delivered, as well as the utility of the same.

In the foregoing, treatment regimens, the subject “in need thereof” is contemplated to embrace, inter alia, patients suffering trauma, undergoing high blood loss surgical procedures, such as hip replacement, or suffering loss of clotting factors due to inherited disorders or other diseases.

As stated above, the present invention is not limited to plasma or blood sources. The present invention (apparatus and fractionation method) may be expanded to fractionation of any protein sample obtained from various sources, including (but not limited to): recombinant sources (e.g., bacterial cultures), manufacturing lines (e.g., synthetic proteins), and biological or medicinal specimens. In this instance, the number of segments in the chamber is only limited by the artisan's desire for fractional resolution. As with the fractionation chamber described above for plasma, the fractionation chamber for this embodiment can have many segments each differing from the foregoing on the basis of the molecular weight cut-off of the membrane bounding that segment. However, the molecular weight cut-off of the membrane should progressively decrease with each subsequent segment downstream.

For example, where the artisan is looking to identify the presence of a transcriptional regulator in a bacterial sample, the artisan may wish to finely resolve proteins within the range of 10-50 kDa, but is not interested in the proteins that are in excess of 50 kDa. In this example, the first segment may have membrane with a molecular weight cut-off of about 50 kDa to remove any larger proteins. The chamber may then contain four additional segments, each with a membrane with a molecular weight cut-off that differs from the one preceding it by 10 kDa. In this manner, the artisan may then take the isolated and concentrated protein fractions and assay for transcriptional regulator activity.

The above written description of the invention provides a manner and process of making and using it such that any person skilled in this art is enabled to make and use the same, this enablement being provided in particular for the subject matter of the appended claims, which make up a part of the original description.

As used herein, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Numerous modifications and variations on the present invention are possible in light of the above teachings. It is, therefore, to be understood that within the scope of the accompanying claims, the invention may be practiced otherwise than as specifically described herein.

1. An apparatus comprising:
a) a plurality of segments; and
b) a membrane at the base of each segment,
wherein each segment has a circular shape with a diameter to height ratio ranging from 10:1 to 20:1,
wherein each segment contains a broad, flat rotor induces complete mixing,
wherein each segment comprises a pressure affecting means,
wherein each segment has an input prior to said membrane and an outlet after said membrane,
wherein each segment is coupled to the input of the subsequent segment through its outlet wherein a pump or regulator system is positioned downstream of the membrane to maintain a pressure difference across the membrane,
wherein the molecular weight cut-off of each membrane is lower than the membrane in the segment upstream thereof.

2. The apparatus of claim 1, wherein said rotor fills at least 70% of the chamber volume.

3. The apparatus of claim 1, wherein said pressure affecting means is a hydraulic pressure affecting means.

4. The apparatus of claim 1, wherein said pressure affecting means maintains a pressure of 200 to 1000 psi.

5. The apparatus of claim 1, wherein said plurality of segments have a steel mesh bound by a steel ring at its base upon which said membrane is located.

6. The apparatus of claim 1, wherein said plurality of segments are made of stainless steel.

7. The apparatus of claim 1, comprising:
a) a first segment;
b) a second segment; and
c) a third segment.

8. The apparatus of claim 7, wherein said the membrane of said first segment has a molecular weight cut-off ranging from 70 kDa to 140 kDa.

9. The apparatus of claim 7, wherein said the membrane of said second segment has a molecular weight cut-off ranging from 40 kDa to 65 kDa.

10. The apparatus of claim 7, wherein said the membrane of said third segment has a molecular weight cut-off ranging from 5 kDa to 15 kDa.

11. A method of fractionating plasma comprising
a) adding plasma to the first segment of the chamber of claim 7;
b) applying a pressure to the solution in the first segment to facilitate transfer of solutes across the membranes;
c) stopping the fractionation after an appropriate time to reach a final transfer equilibrium; and
d) recovering samples from each of the first, second, and third segments,
wherein each of (a)-(d) are performed under sterile conditions.

12. The method of claim 11, further comprising:
e) further concentrating the sample recovered from the first segment by cryoconcentration.

13. The method of claim 11, wherein said plasma is obtained from a subject selected from the group consisting of human, horse, dog, cat, sheep, cattle, pig, primate, and mouse.

14. The method of claim 11, wherein said plasma is obtained from a human.

15. The method of claim 11, wherein said pressure is a hydraulic pressure.

16. The method of claim 11, wherein said pressure ranges from 200 to 1000 psi.

17. The method of claim 11, further comprising stirring at a rotor speed of no more than 50 rpm during (b).

18. An isolated immune factor and clotting factor concentrate obtained by the method of claim 11 and recovered from the first segment of the chamber.

19. The isolated immune factor and clotting factor concentrate of claim 18, wherein said isolated immune factor and clotting factor concentrate is substantially pure.

20. An isolated albumin concentrate obtained by the method of claim 11 and recovered from the second segment of the chamber.

21. The isolated albumin concentrate of claim 20, wherein said isolated albumin concentrate is substantially pure.

22. The isolated albumin concentrate of claim 20, wherein said isolated albumin concentrate contains clotting factor X.

23. An isolated concentrate comprising small plasma proteins and sugars obtained by the method of claim 1 1 and recovered from the third segment of the chamber.

24. A method of promoting clotting in a subject in need thereof comprising administering to said subject an effective amount of the immune factor and clotting factor concentrate of claim 18.

25. The method of claim 24, wherein said administering is intravenously.

26. The method of claim 24, wherein said administering comprises mixing the clotting factor concentrate with thrombin and spraying, pouring, or extruding the mixture on open wound areas to induce hemostasis.

27. The apparatus of claim 1, comprising:
a) a first segment;
b) a second segment;
c) a third segment; and
d) a fourth segment.

28. The apparatus of claim 27, wherein said the membrane of said first segment has a molecular weight cut-off ranging from 300 kDa to 500 kDa.

29. The apparatus of claim 27, wherein said the membrane of said second segment has a molecular weight cut-off ranging from 70 kDa to 140 kDa.

30. The apparatus of claim 27, wherein said the membrane of said third segment has a molecular weight cut-off ranging from 40 kDa to 65 kDa.

31. The apparatus of claim 27, wherein said the membrane of said fourth segment has a molecular weight cut-off ranging from 5 kDa to 15 kDa.

32. A method of fractionating plasma comprising
a) adding plasma to the first segment of the chamber of claim 27;
b) applying a pressure to the solution in the first segment to facilitate transfer of solutes across the membranes;
c) stopping the fractionation after an appropriate time to reach a final transfer equilibrium; and
d) recovering samples from each of the first, second, and third segments, wherein each of (a)-(d) are performed under sterile conditions.

33. The method of claim 32, further comprising:
e) further concentrating the sample recovered from the first segment by cryoconcentration.

34. The method of claim 32, wherein said plasma is obtained from a subject selected from the group consisting of human, horse, dog, cat, sheep, cattle, pig, primate, and mouse.

35. The method of claim 32, wherein said plasma is obtained from a human.

36. The method of claim 32, wherein said pressure is a hydraulic pressure.

37. The method of claim 32, wherein said pressure ranges from 200 to 1000 psi.

38. The method of claim 32, further comprising stirring at a rotor speed of no more than 50 rpm during (b).

39. An isolated immune factor concentrate obtained by the method of claim 32 and recovered from the first segment of the chamber

40. The isolated immune factor concentrate of claim 39, wherein said isolated immune factor concentrate is substantially pure.

41. An isolated clotting factor concentrate obtained by the method of claim 32 and recovered from the second segment of the chamber.

42. The isolated clotting factor concentrate of claim 41, wherein said isolated clotting factor concentrate is substantially pure.

43. An isolated albumin concentrate obtained by the method of claim 32 and recovered from the third segment of the chamber.

44. The isolated albumin concentrate of claim 43, wherein said isolated albumin concentrate is substantially pure.

45. The isolated albumin concentrate of claim 43, wherein said isolated albumin concentrate contains clotting factor X.

46. An isolated concentrate comprising small plasma proteins and sugars obtained by the method of claim 32 and recovered from the fourth segment of the chamber.

47. A method of promoting clotting in a subject in need thereof comprising administering to said subject an effective amount of the clotting factor concentrate of claim 41.

48. The method of claim 47, wherein said administering is intravenously.

49. The method of claim 47, wherein said administering comprises mixing the clotting factor concentrate with thrombin and spraying, pouring, or extruding the mixture on open wound areas to induce hemostasis.
